(19) United States
(12) Patent Application Publication
(10) Pub. No.: US 2014/0186407 A9
(43) Pub. Date: Jul. 3, 2014

(54) C-Met Modulator Pharmaceutical Compositions

(71) Applicant: Jo Ann Wilson, San Francisco, CA (US)

(72) Inventor: Jo Ann Wilson, San Francisco, CA (US)

(73) Assignee: Exelixis Inc., South San Francisco, CA (US)

(21) Appl. No.: 13/810,546

(22) PCT Filed: Jul. 18, 2011
(86) PCT No.: PCT/US11/44382

§ 371 Date: Aug. 27, 2013

Related U.S. Application Data
(60) Provisional application No. 61/365,261, filed on Jul. 16, 2010.

(51) Int. Cl. A61K 31/5377; A61K 45/06; A61K 9/00

(52) U.S. Cl. CPC A61K 31/5377; A61K 9/00; A61K 45/06
USPC 424/400; 514/235.2; 264/330; 427/2.14

(57) ABSTRACT

Pharmaceutical compositions and unit dosage forms comprising Compound I are disclosed.

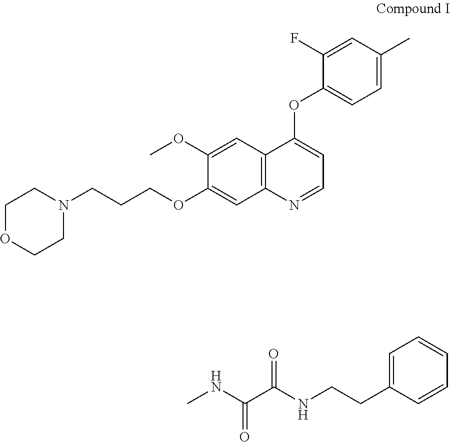

### CROSS-REFERENCE TO RELATED APPLICATION

This application claims priority to U.S. application Ser. No. 61/365,261, filed Jul. 16, 2010, the entire contents of each of which are incorporated herein by reference.

### FIELD OF THE INVENTION

Traditionally, dramatic improvements in the treatment of cancer are associated with identification of therapeutic agents acting through novel mechanisms. One mechanism that can be exploited in cancer treatment is the modulation of protein kinase activity. Signal transduction through protein kinase activation is responsible for many of the characteristics of tumor cells. Protein kinase signal transduction is particularly relevant in, for example, renal cancer, gastric cancer, head and neck cancers, lung cancer, breast cancer, prostate cancer, colorectal cancers, and hepatocellular carcinoma, as well as in the growth and proliferation of brain tumor cells.

Protein kinases can be categorized as receptor type or non-receptor type. Receptor-type tyrosine kinases are comprised of a large number of transmembrane receptors with diverse biological activity. For a detailed discussion of the receptor-type tyrosine kinases, see Plowman et al., DN&P 7(6): 334-339, 1994. Since protein kinases and their ligands play critical roles in various cellular activities, deregulation of protein kinase enzymatic activity can lead to altered cellular properties, such as uncontrolled cell growth that is associated with cancer. In addition to oncological indications, altered kinase signaling is implicated in numerous other pathological diseases, including, for example, immunological disorders, cardiovascular diseases, inflammatory diseases, and degenerative diseases. Protein kinases are therefore attractive targets for small molecule drug discovery. Particularly attractive targets for small-molecule modulation with respect to antiangiogenic and antiproliferative activity include receptor type tyrosine kinases c-Met, KDR, c-Kit, Axl, flt-3, and flt-4.

The kinase c-Met is the prototypic member of a subfamily of heterodimeric receptor tyrosine kinases (RTKs), which include Met, Ron, and Sea. The endogenous ligand for c-Met is the hepatocyte growth factor (HGF), a potent inducer of angiogenesis. Binding of HGF to c-Met induces activation of the receptor via autophosphorylation resulting in an increase of receptor dependent signaling, which promotes cell growth and invasion. Anti-HGF antibodies or HGF antagonists have been shown to inhibit tumor metastasis in vivo (See Maulik et al Cytokine & Growth Factor Reviews 2002 13, 41-59). c-Met overexpression has been demonstrated on a wide variety of tumor types, including breast, colon, renal, lung, squamous cell myeloid leukemia, hemangiomas, melanomas, astrocytomas, and glioblastomas. Additionally, activating mutations in the kinase domain of c-Met have been identified in hereditary and sporadic renal papilloma and squamous cell carcinoma. (See, e.g., Maulik et al., Cytokine & growth Factor reviews 2002 13, 41-59; Longati et al., Curr Drug Targets 2001, 2, 41-55; Funakoshi et al., Clinica Chimica Acta 2003 1-23).

Inhibition of epidermal growth factor (EGF), vascular endothelial growth factor (VEGF), and ephrin signal transduction will prevent cell proliferation and angiogenesis, both of which are key cellular processes needed for tumor growth and survival (Matter A., Drug Disc. Technol. 2001 6, 1005-1024). Kinase KDR (refers to kinase insert domain receptor tyrosine kinase) and flt-4 (fms-like tyrosine kinase-4) are both VEGF receptors. EGF and VEGF receptors are desirable targets for small molecule inhibition. All members of the VEGF family stimulate cellular responses by binding to tyrosine kinase receptors (the VEGFRs) on the cell surface, which causes them to dimerize and become activated through transphosphorylation. The VEGF receptors have an extracellular portion with immunoglobulin-like domains, a single transmembrane spanning region, and an intracellular portion containing a split tyrosine-kinase domain. VEGF binds to VEGFR-1 and VEGFR-2. VEGFR-2 is known to mediate almost all of the known cellular responses to VEGF.

Kinase c-Kit (also called stem cell factor receptor or steel factor receptor) is a type 3 receptor tyrosine kinase (RTK) that belongs to the platelet-derived growth factor receptor subfamily. Overexpression of c-Kit and c-Kit ligand has been described in variety of human diseases, including human gastrointestinal stromal tumors, mastocytosis, germ cell tumors, acute myeloid leukemia (AML), NK lymphoma, small-cell lung cancer, neuroblastomas, gynecological tumors, and colon carcinoma. Moreover, elevated expression of c-Kit may also relate to the development of neoplasia associated with neurofibromatosis type 1 (NF-1), mesenchymal tumors GISTs, and mast cell disease, as well as other disorders associated with activated c-Kit.

Kinase Flt-3 (fms-like tyrosine kinase-3) is constitutively activated via mutation, either in the juxtamembrane region or in the activation loop of the kinase domain, in a large proportion of patients with AML (acute myeloid leukemia) (See Reilly, Leuk. Lymphoma, 2003, 44: 1-7).

Accordingly, small-molecule compounds that specifically inhibit, regulate, and/or modulate the signal transduction of kinases, including c-Met, VEGFR2, KDR, c-Kit, Axl, flt-3, and flt-4, are particularly desirable as a means to treat or prevent disease states that are associated with abnormal cell proliferation and angiogenesis. One such small-molecule is Compound I, which has the chemical structure:

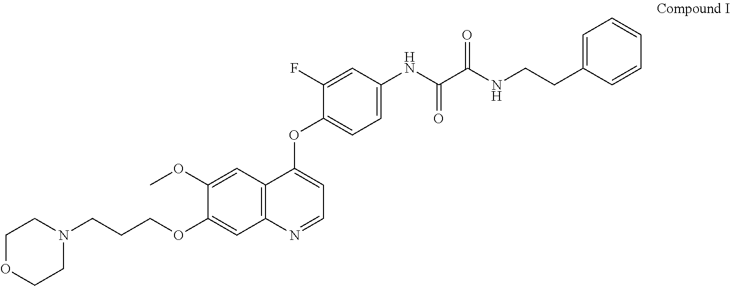 [SMILES: COc1cc2c(Oc3ccc(NC(=O)C(=O)NCCc4ccccc4)cc3F)ccnc2cc1OCCCN1CCOCC1]

Compound 1 is disclosed and claimed in WO2005/030140, the entire contents of which are herein incorporated by reference. WO2005/030140 describes the synthesis of Compound I (Table 1, Compound 135, Example 41) and discloses the therapeutic activity of this molecule to inhibit, regulate, and/or modulate the signal transduction of kinases (Assays, Table 4, entry 137). An alternative synthesis of Compound I is disclosed in PCT/US2009/066747.

Although therapeutic efficacy is the primary concern for a therapeutic agent, the pharmaceutical composition can be equally important to its development. Generally, drug developers endeavor to discover a pharmaceutical composition that possesses desirable properties, such as satisfactory water-solubility (including rate of dissolution), storage stability, hygroscopicity, and reproducibility, all of which can impact the processability, manufacture, and/or bioavailability of the drug. Accordingly, discovery of pharmaceutical compositions that possess some or all of these desired properties is vital to drug development.

### SUMMARY OF THE INVENTION

These and other needs are met by the present disclosure, which is directed to a pharmaceutical composition comprising Compound I as provided in Table 1.

TABLE 1

| Ingredient | (% w/w) |
|---|---|
| Compound I | 31.68 |
| Microcrystalline Cellulose | 38.85 |
| Lactose anhydrous | 19.42 |
| Hydroxypropyl Cellulose | 3.00 |
| Croscarmellose Sodium | 3.00 |
| Total Intra-granular | 95.95 |
| Silicon dioxide, Colloidal | 0.30 |
| Croscarmellose Sodium | 3.00 |
| Magnesium Stearate | 0.75 |
| Total | 100.00 |

The disclosure is also directed to a pharmaceutical composition comprising Compound I as provided in Table 2.

TABLE 2

| Ingredient | (% w/w) |
|---|---|
| Compound I | 25.0-33.3 |
| Microcrystalline Cellulose | q.s |
| Hydroxypropyl Cellulose | 3 |
| Poloxamer | 0-3 |
| Croscarmellose Sodium | 6.0 |
| Colloidal Silicon Dioxide | 0.5 |
| Magnesium Stearate | 0.5-1.0 |
| Total | 100 |

The disclosure is further directed to a pharmaceutical composition comprising Compound I as provided in Table 3.

TABLE 3

| | Theoretical Quantity |
|---|---|
| Ingredient | (mg/unit dose) |
| Compound I | 100.0 |
| Microcrystalline Cellulose PH-102 | 155.4 |
| Lactose Anhydrous 60M | 77.7 |
| Hydroxypropyl Cellulose, EXF | 12.0 |
| Croscarmellose Sodium | 24 |
| Colloidal Silicon Dioxide | 1.2 |
| Magnesium Stearate (Non-Bovine) | 3.0 |
| Opadry Yellow | 16.0 |
| Total | 416 |

In one aspect, Compound I is present in Tables 1, 2, and 3 as the L-malate salt.

The disclosure is also directed to a process of preparing a pharmaceutical composition according to Tables 1, 2, or 3.

The disclosure is further directed to a method for treating cancer, comprising administering to a patient in need of such treatment a pharmaceutical composition according to Tables 1, 2, or 3. The disclosure is also directed to a method for treating cancer, comprising administering to a patient in need of such treatment a pharmaceutical composition according to Tables 1, 2, or 3 in combination with another therapeutic agent.

In these and other treatment aspects, the cancers to be treated include the cancers disclosed in WO2005/030140, including pancreatic cancer, kidney cancer, liver cancer, prostate cancer, gastric cancer, gastroesophageal cancer, melanoma, lung cancer, breast cancer, thyroid cancer, and astrocytic tumors. More particularly, the cancers include pancreatic cancer, hepatocellular carcinoma (HCC), renal cell carcinoma, castration-resistant prostate cancer (CRPC), gastric or gastroesophageal junction cancer, melanoma, small cell lung cancer (SCLC), ovarian cancer, primary peritoneal or fallopian tube carcinoma, estrogen receptor positive breast cancer, estrogen receptor/progesterone receptor/HER2-negative (triple-negative) breast cancer, inflammatory (regardless of receptor status) breast cancer histology, non-small cell lung cancer (NSCLC), and medullary thyroid cancer.

### DETAILED DESCRIPTION

The disclosure is directed to a pharmaceutical formulation comprising Compound I and pharmaceutically acceptable filler, binder, disintegrant, glidant, and lubricant, and optionally a film coating material, each of which are described in greater detail in the following paragraphs. Examples of pharmaceutically acceptable fillers, binders, disintegrants, glidants, lubricants, and film coatings are set forth below and are described in more detail in the Handbook of Pharmaceutical Excipients, Second Edition, Ed. A. Wade and P. J. Weller, 1994, The Pharmaceutical Press, London, England. The term excipient as used herein refers to inert materials which impart satisfactory processing and compression characteristics into the formulation or impart desired physical characteristics to the finished table.

### Compound I Pharmaceutical Composition

The Compound I pharmaceutical composition is a tablet comprising Compound I and excipients selected from the group consisting of a filler, a binder, a disintegrant, a glidant, and a lubricant, and optionally may be coated or uncoated.

### Compound I

In one embodiment, the pharmaceutical composition comprises Compound I as the free base.

In another embodiment, the pharmaceutical composition comprises Compound I as a hydrate.

In another embodiment, the pharmaceutical composition comprises Compound I as a salt. “Pharmaceutically acceptable salt” refers to those salts that retain the biological effectiveness of the free bases and that are not biologically or otherwise undesirable, formed with inorganic acids, such as hydrochloric acid, hydrobromic acid, sulfuric acid, nitric acid, phosphoric acid, and the like, or mixtures thereof, as well as organic acids, such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, or mixtures thereof.

In another embodiment, the Compound I or the Compound I pharmaceutically acceptable salt is in amorphous or substantially amorphous form. “Substantially amorphous” means that more than 50 percent of the Compound I or the Compound I pharmaceutically acceptable salt is amorphous.

In another embodiment, the Compound I or the Compound I pharmaceutically acceptable salt is in crystalline or substantially crystalline form. “Substantially crystalline” means that more than 50 percent of the Compound I or the Compound I pharmaceutically acceptable salt are crystalline.

### Filler

As indicated above, the pharmaceutical composition containing Compound I comprises a filler. Fillers are inert ingredients added to adjust the bulk in order to produce a size practical for compression. Examples of fillers include sodium starch glycolate, corn starch, talc, sucrose, dextrose, glucose, lactose, xylitol, fructose, sorbitol, calcium phosphate, calcium sulfate, calcium carbonate, and the like, or mixtures thereof. Microcrystalline cellulose may also be used as a filler and may be any suitable form of microcrystalline cellulose as is known and used in the tabletting art. Preferably, a mixture of lactose and microcrystalline cellulose is used as the filler. In one embodiment, the lactose is anhydrous lactose sold as Lactose 60M, which is readily commercially available from a number of suppliers. In one embodiment, the microcrystalline cellulose is Avicel PH-102, which is also commercially available.

Preferably, filler(s) are present in an amount of from about 50 to about 70 percent, and more preferably from about 57 to about 67 percent, by weight on a solids basis of the directly compressible formulation. Preferably, lactose is present in an amount of from about 18 to 22 percent by weight. Preferably, the microcrystalline cellulose is present in an amount of from about 38 to 40 percent by weight.

### Binder

The pharmaceutical composition containing Compound I also comprises a binder. Binders are added to powders to impart cohesive qualities to the powder, which allows the compressed tablet to retain its integrity. The binder can be any pharmaceutically acceptable binder available in the tabletting art, such as acacia, alginic acid, carbomer, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, zein, and the like, or mixtures thereof.

The preferred binder is hydroxypropyl cellulose preferably in an amount of from about 2 to about 4 percent by weight on a solid basis of the directly compressible formulation. In one embodiment, the hydroxypropyl cellulose is commercially available Klucel EXF.

### Disintegrant

The pharmaceutical composition containing Compound I also comprises a disintegrant. A disintegrant is a substance or a mixture of substances added to facilitate breakup or disintegrate after administration. The disintegrant may be any pharmaceutically acceptable disintegrant available in the tabletting art, including alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, powdered cellulose, pregelatinized starch, sodium alginate, starch, and the like, or mixtures thereof.

The preferred disintegrant is croscarmellose sodium, in an amount of from about 4 to about 8 percent by weight, on a solid basis of the directly compressible formulation. In one embodiment, the croscarmellose sodium is commercially available Ac-Di-Sol.

### Glidant

The pharmaceutical composition containing Compound I also comprises a glidant. The glidant may be any pharmaceutically acceptable glidant which contributes to the compressibility, flowability, and homogeneity of the formulation and which minimizes segregation and does not significantly interfere with the release mechanism of the binders as set forth above. Preferably, the glidant is selected to improve the flow of the formulation. Silicon dioxide, particularly colloidal silicon dioxide, is preferred as a glidant.

The glidant is used in an amount of from about 0.2 to about 0.6 percent by weight on a solid basis of the directly compressible formulation.

### Lubricant

The pharmaceutical composition containing Compound I also comprises a lubricant. Lubricants are employed to prevent adhesion of the tablet material to the surface of dyes and punches. The lubricant may be any pharmaceutically acceptable lubricant which substantially prevents segregation of the powder by contributing to homogeneity of the formulation and which exhibits good flowability. Preferably, the lubricant functions to facilitate compression of the tablets and ejection of the tablets from the die cavity. Such lubricants may be hydrophilic or hydrophobic, and examples include magnesium stearate, Lubritab®, stearic acid, talc, and other lubricants known in the art or to be developed which exhibit acceptable or comparable properties, or mixtures thereof. Examples of lubricants include calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, and the like, or mixtures thereof.

The lubricant should be selected to aid in the flow of the powder in the hopper and into the die. Magnesium stearate exhibits excellent properties in combination with the other preferred excipients of the formulation. Magnesium stearate contributes to reducing friction between the die wall and tablet formulation during compression, as well as to the easy ejection of the Compound I tablets. It also resists adhesion to punches and dies.

Preferably, the lubricant is magnesium stearate (non-bovine) used in an amount of from about 0.5 to about 1.0 percent by weight on a solid basis of the directly compressible formulation.

### Film Coating

The pharmaceutical composition containing Compound I also comprises an optional film coating. The film coat concentration can be about 1 to about 10 percent by weight on a solid basis of the directly compressible formulation. Film coating suspensions may include combinations of the following components: hypromeollose, carboxymethylcellulose sodium, carnauba wax, cellulose acetate phthalate, cetyl alcohol, confectioner's sugar, ethyl cellulose, gelatin, hydroxyethyl cellulose, hydroxypropyl cellulose, hydroxypropyl methylcellulose, liquid glucose, maltodextrin, methyl cellulose, microcrystalline wax, Opadry and Opadry II, polymethacrylates, polyvinyl alcohol, shellac, sucrose, talc, titanium dioxide, and zein.

Preferably the film coating comprises commercial available Opadry Yellow.

In one embodiment, the tablet composition comprises
30-32 percent by weight of Compound I in at least one of the forms disclosed herein;
50-70 percent by weight of a filler;
2-4 percent by weight of a binder;
4-8percent by weight a disintegrant; and
0.2-0.6 percent by weight of a glidant; and 0.5-1 percent by weight of a lubricant.

In another embodiment, the tablet composition comprises
30-32 percent by weight of Compound I in at least one of the forms disclosed herein;
50-70 percent by weight of a filler;
2-4 percent by weight of a binder;
4-8percent by weight a disintegrant; and
0.2-0.6 percent by weigh of a glidant; and 0.5-1 percent by weight of a lubricant;
   wherein the composition is coated.

In another embodiment, the tablet composition comprises:

| Component | Weight/Weight Percent |
|---|---|
| Compound I | 25-29 |
| Microcrystalline Cellulose | q.s. |
| Lactose Anhydrous | 40-44 |
| Hydroxypropyl Cellulose | 2-4 |
| Croscarmellose Sodium | 2-8 |
| Colloidal Silicon Dioxide | 0.1-0.4 |
| Magnesium Stearate | 0.7-0.9 |
| Total | 100 |

In another embodiment, the tablet compositions of this disclosure contain from 10 to about 200 mg of Compound I in at least one of the forms described herein. In another embodiment, the tablet compositions of this disclosure contain from 20 to 100 mg of Compound I. In another embodiment, the tablet compositions contain 20, 25, 50, 60, 75, 80, or 100 mg of Compound I.

In other embodiments, the tablet compositions are summarized in Tables 1, 2, and 3. The compound I used in these and other compositions disclosed herein is the L-malate salt Compound I. In the tables, the weight of Compound I refers to the amount of N-[4-[(6,7-Dimethoxy-4-quinolnyl)oxy]phenyl]-N′-(4-fluorophenyl)-1,1-cyclopropanedicarboxamide in the tablet. The skilled artisan will recognize that a certain amount of the Compound I L-malate salt is required to provide the weights listed in the tables. Thus, for example, in Table 3, 126.7 mg of Compound I L-malate salt is required to provide 100 mg of Compound I. Proportionally smaller or larger amounts of Compound I L-malate salt are required for tablet compositions containing less or more Compound I.

### Process

In another aspect, the disclosure is directed to a process for making pharmaceutical formulations comprising Compound I. In one embodiment, the formulation is a tablet formulation.

In another embodiment, the process comprises mixing Compound I with one or more of the pharmaceutical excipients. The mixture is then taken up in an aqueous solution containing a binder to form a binder solution. The binder solution is granulated using a granulation technique known in the art. For example, the granulation method may comprise wet high shear granulation using a wet high shear granulator. The resulting wet granules are then screened and dried using fluid bed drying or the like. The dried granules are then milled. The resulting dry milled granules are then mixed with a glidant and a disintegrant to form an extra-granular blend. A lubricant is then blended into the extraganular blend to form the final blend. The final blend is subsequently compressed to form the compressed tablet, which may be film coated.

More particularly, the process comprises delumping Compound I as needed prior to mixing with the excipients. Delumping ensures that the Compound I mixes homogeneously with the other excipients during the formulation process. Delumped Compound I is then mixed with microcrystalline cellulose, such as Avicel PH 102, lactose (anhydrous, 60M), and croscarmellose sodium. This mixture is then combined with EXF grade hydroxypropoyl cellulose in water to form a binder solution, which is then wet high shear granulated. The resulting wet granules are wet screened and then fluid bed dried according to methods available to the skilled artisan. The resulting dried granules are milled and combined with colloidal silicon dioxide and croscarmellose sodium. Magnesium stearate is added to the mixture. This final blend is then ready for tablet compression. The resulting uncoated core tablets are subsequently film coated. The film coating comprises Opadry Yellow, which contains hypromellose, titanium dioxide, triacetin, and iron oxide yellow.

More particularly, the formulation process comprises:
a) Delumping unmilled Compound I;
b) Premixing the delumped Compound I with Avicel PH102, lactose anhydrous 60M, and croscarmellose sodium to form a binder solution;
c) Wet high shear granulation of the binder solution to produce wet granules;
d) Wet screening of the wet granules to produce wet screened granules;
e) Fluid bed drying of the wet screened granules to produce dried granules;
f) Dry milling of the dried granules to produce dried milled granules;
g) Blending the dried milled granules with colloidal silicon and croscarmellose to produce an extragranular blend;
h) Lubricant blending of the extragranular blend and magnesium stearate to produce a final blend;
i) Tablet compression of the final blend to form an uncoated core tablet; and
j) Film coating of the uncoated core tablet.

### Methods of Treatment

Another aspect of this disclosure relates to a method of treating cancer, as discussed above, where the cancer treated is stomach cancer, esophageal carcinoma, kidney cancer, liver cancer, ovarian carcinoma, cervical carcinoma, large bowel cancer, small bowel cancer, brain cancer (including astrocytic tumor, which includes glioblastoma, giant cell glioblastoma, gliosarcoma, and glioblastoma with oligodendroglial components), lung cancer (including non-small cell lung cancer), bone cancer, prostate carcinoma, pancreatic carcinoma, skin cancer, bone cancer, lymphoma, solid tumors, Hodgkin's disease, non-Hodgkin's lymphoma, or thyroid cancer (including medullary thyroid cancer). More particularly, the cancer is pancreatic cancer, hepatocellular carcinoma (HCC), renal cell carcinoma, castration-resistant prostate cancer (CRPC), gastric or gastroesophageal junction cancer, melanoma, small cell lung cancer (SCLC), ovarian cancer, primary peritoneal or fallopian tube carcinoma, estrogen receptor positive breast cancer, estrogen receptor/progesterone receptor/HER2-negative (triple-negative) breast cancer, inflammatory (regardless of receptor status) breast cancer, non-small cell lung cancer (NSCLC), or medullary thyroid cancer.

Tyrosine kinase inhibitors have also been used to treat non-small cell lung cancer (NSCLC). Gefitinib and erlotinib are angiogenesis inhibitors that target receptors of an epidermal growth factor called tyrosine kinase. Erlotinib and Gefitinib are currently being used for treating NSCLC. Another aspect of this disclosure relates to a method of treating non-small cell lung cancer (NSCLC) comprising administering to the subject in need of the treatment a therapeutically effective amount of Compound I in at least one of the forms described herein, pharmaceutically formulated as described herein, optionally in combination with Erlotinib or Gefitinib. In another embodiment, the combination is with Erlotinib.

In another embodiment, the cancer is non-small cell lung cancer (NSCLC), and the method comprises administering to the subject in need of the treatment a therapeutically effective amount of Erlotinib or Gefitinib in combination with at least one of the forms of Compound I in at least one of the forms described herein pharmaceutically formulated as described herein. The method of treatment may be practiced by administering a tablet formulation of at Compound I in at least one of the forms described herein, pharmaceutically formulated as described herein.

Another aspect of this disclosure relates to a method of treating an astrocytic tumor (which includes glioblastoma, giant cell glioblastoma, gliosarcoma, and glioblastoma with oligodendroglial components) comprising administering to the subject in need of the treatment a therapeutically effective amount of Compound I in at least one of the forms described herein, pharmaceutically formulated as described herein.

Another aspect of this disclosure relates to a method of treating thyroid cancer (including medullary thyroid cancer) comprising administering to the subject in need of the treatment Compound I in at least one of the forms described herein, pharmaceutically formulated as described herein. The amount administered can be a therapeutically effective amount.

Another aspect of this disclosure relates to a method of treating pancreatic cancer comprising administering to the subject in need of the treatment Compound I in at least one of the forms described herein, pharmaceutically formulated as described herein. The amount administered can be a therapeutically effective amount.

Another aspect of this disclosure relates to a method of treating castration resistant prostate cancer comprising administering to the subject in need of the treatment Compound I in at least one of the forms described herein, pharmaceutically formulated as described herein. The amount administered can be a therapeutically effective amount.

Another aspect of this disclosure relates to a method of treating hepatoceular carcinoma comprising administering to the subject in need of the treatment Compound I in at least one of the forms described herein, pharmaceutically formulated as described herein. The amount administered can be a therapeutically effective amount.

Another aspect of this disclosure relates to a method of treating renal cell carcinoma comprising administering to the subject in need of the treatment Compound I in at least one of the forms described herein, pharmaceutically formulated as described herein. The amount administered can be a therapeutically effective amount.

Another aspect of this disclosure relates to a method of treating breast cancer, including estrogen receptor positive breast cancer, estrogen receptor/progesterone receptor/HER2-negative (triple-negative) breast cancer, or inflammatory (regardless of receptor status) breast cancer, comprising administering to the subject in need of the treatment Compound I in at least one of the forms described herein, pharmaceutically formulated as described herein. The amount administered can be a therapeutically effective amount.

Another aspect of this disclosure relates to a method of treating diseases or disorders associated with uncontrolled, abnormal, and/or unwanted cellular activities. The method comprises administering to the subject in need of the treatment Compound I in at least one of the forms described herein, pharmaceutically formulated as described herein. The amount administered can be a therapeutically effective amount.

A “therapeutically effective amount of the active compounds”, or a crystalline or amorphous form of the active compound(s) to inhibit, regulate, and/or modulate the signal transduction of kinases (discussed here concerning the pharmaceutical compositions) refers to an amount sufficient to treat a patient suffering from any of a variety of cancers associated with abnormal cell proliferation and angiogenesis. A therapeutically effective amount according to this disclosure is an amount therapeutically useful for the treatment or prevention of the disease states and disorders discussed herein. Compound I possess therapeutic activity to inhibit, regulate, and/or modulate the signal transduction of kinases such as described in WO2005/030140.

The actual amount required for treatment of any particular patient will depend upon a variety of factors, including the disease state being treated and its severity; the specific pharmaceutical composition employed; the age, body weight, general health, sex, and diet of the patient; the mode of administration; the time of administration; the route of administration; the rate of excretion of the active compound(s), or a crystalline form of the active compound(s), according to this disclosure; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173, 2001, which is incorporated herein by reference. The active compound(s), or a crystalline form of active compound(s), according to this disclosure, and pharmaceutical compositions comprising them, may be used in combination with anticancer or other agents that are generally administered to a patient being treated for cancer. They may also be co-formulated with one or more of such agents in a single pharmaceutical composition.

The disclosure is further illustrated by the following examples, which are not to be construed as limiting the disclosure in scope or spirit to the specific procedures described in them.

Unless specified otherwise, the starting materials and various intermediates may be obtained from commercial sources, prepared from commercially available organic compounds, or prepared using well-known synthetic methods.

### EXAMPLES

Compound I can be prepared according to Scheme 1.

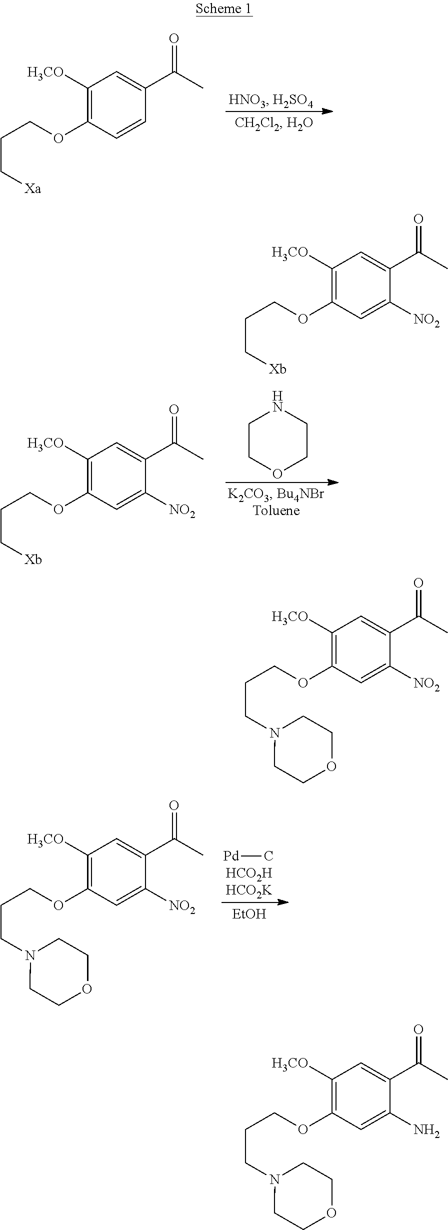

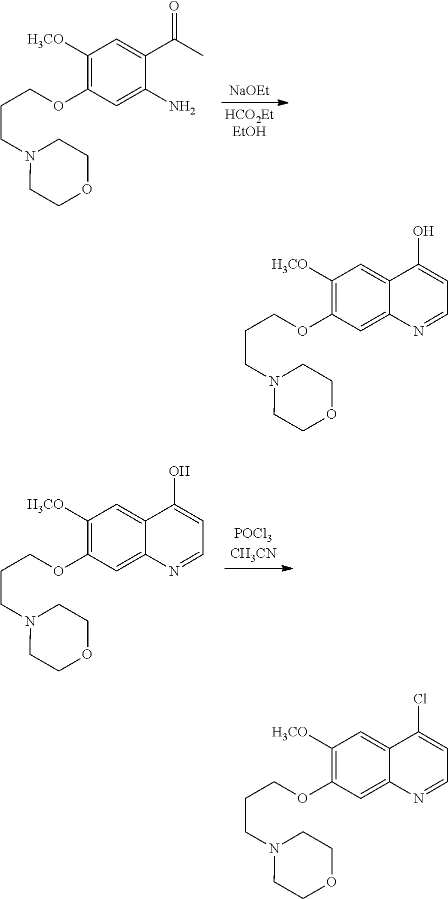 [SMILES: COc1cc(C(C)=O)c(N)cc1OCCCN1CCOCC1 ; COc1cc2c(O)ccnc2cc1OCCCN1CCOCC1 ; COc1cc2c(O)ccnc2cc1OCCCN1CCOCC1 ; "HCO2EtEtOH" ; "NaOEt" ; COc1cc2c(Cl)ccnc2cc1OCCCN1CCOCC1]

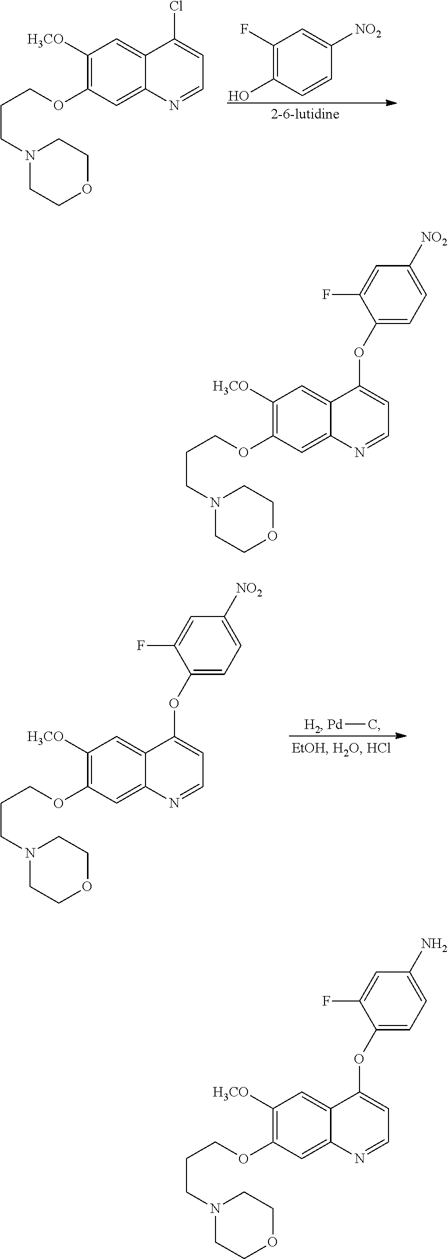

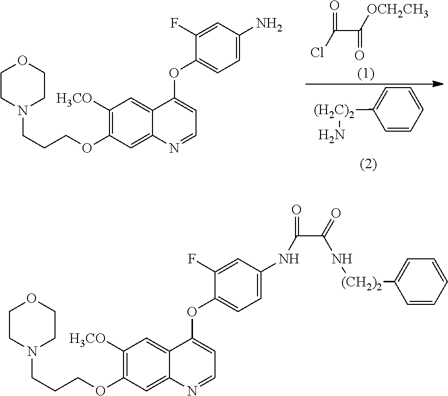

Xa and Xb in Scheme 1 above are each Br or Cl. For the names of the intermediates described within the description of Scheme 1 below, Xa and Xb are both referred to as halo in these names, wherein this halo group for these intermediates is meant to mean either Br or Cl. This definition of halo, which is applicable only to these intermediates in the description of Scheme 1 below, is not meant to change the definition of halo in the definitions section.

### Preparation of 1-[5 methoxy-4(3-halo propoxy)-2 nitro-phenyl]-ethanone

A pre-mixed solution of water (80 L) and concentrated sulfuric acid, 96% (88 L), cooled to approximately 5° C., was charged to a reactor containing to the solution of 1-[4-(3-halo propoxy)-3-methoxy phenyl]ethanone (both of which are commercially available) at a rate such that the batch temperature did not exceed approximately 18° C. The resulting solution was cooled to approximately 5° C., and 65% nitric acid (68 L) was added at a rate such that batch temperature did not exceed approximately 10° C. HPLC analysis was used to determine when the reaction was complete. Methylene chloride (175 L) was charged to a separate reactor containing cooled water (1800 L; by dissolving 450 Kg of ice in 1500 of water). The acidic reaction mixture was then added into this mixture. The methylene chloride layer was separated, and the aqueous layer was back extracted with methylene chloride (78 L). The combined methylene chloride layers were washed with two portions of a solution of aqueous sodium bicarbonate followed by water (50 L) and then concentrated by vacuum distillation. 1-Butanol (590 L) was added, and the mixture was again concentrated by vacuum distillation. The resulting solution was stirred at approximately 20° C., during which time the product crystallized. The solids were recovered by filtration and washed with heptane (100 L) to afford the title compound (89.8 kg wet). The mother liquor was concentrated, and the resulting solid was filtered and washed with n-heptane (45 L) to afford a second crop of the title compound (25 kg wet). Both product crops were combined and dried in a tumble drier at 35° C. to yield product (99.7 kg; 25.6% LOD), which was used directly in the next step without further drying. Three production batches were made.

¹HNMR (400 MHz, DMSO-d6): δ. 7.69 (s, 1H), 7.24 (s, 1H); 4.23 (m, 2H), 3.94 (s, 3H), 3.78 (t)-3.65 (t) (2H), 2.51 (s, 3H), 2.30-2.08 (m, 2H) LC/MS Calcd for [M(Cl)+H]⁺ 288.1, found 288.0; Calcd for [M(Br)+H]⁺ 332.0, 334.0, found 331.9, 334.0.

### Preparation of 1-[5-methoxy-4-(3-morpholin-4-yl-propoxy)-2-nitro-phenyl]-ethanone

The solvent wet cake isolated in the previous step (82.8 kg wet; 74.2 kg dry calc.) was dissolved in toluene (390 L). A solution of sodium iodide (29.9 kg) and potassium carbonate (53.4.0 kg) dissolved in water (170 L) was added to this solution, followed by tetrabutylammonium bromide (8.3 kg) and morpholine (67 L). The resulting two-phase mixture was heated to approximately 85° C. for about 10 hours (the reaction completion was tested by an in-process HPLC). The mixture was then cooled to ambient temperature. The organic layer was separated. The aqueous layer was back extracted with toluene (103 L). The combined toluene layers were washed sequentially with two portions of 5% sodium thiosulfate (259 L each) [sodium thiosulfate (26.8 kg) dissolved in water (550 L)], followed by two portions of aqueous NaCl (256 L; NaCl; 15 kg dissolved in water; 300 L). The resulting solution was concentrated under vacuum, and n-heptane (340 L) was then charged. The resulting slurry was filtered and washed with n-heptane (75 L) to yield the title compound (92% AUC, HPLC82.8 wet; 67.2 dry calculated) which was used in the next step without drying. Four manufacturing batches were carried out for this step.

¹ HNMR (400 MHz, DMSO-d6): δ. 7.64 (s, 1H), 7.22 (s, 1H), 4.15 (t, 2H), 3.93 (s, 3H), 3.57 (t, 4H), 2.52 (s, 3H), 2.44-2.30 (m, 6H), 1.90 (quin, 2H); LC/MS Calcd for [M+H]⁺ 339.2, found 339.2.

### Preparation of 1-[2-amino-5-methoxy-4-(3-morpholin-4-yl-propoxy)-phenyl]-ethanone

The product from the previous step (30.3 kg), followed by ethanol (22 L) and 10% palladium on carbon (Pd—C; 50% water wet, 2.75 kg), was charged to a reactor. The resulting slurry was heated to approximately 48° C., and a solution of formic acid (12 L), potassium formate (22.6 kg), and water (30.8 L) was added. When the addition was complete and the reaction was deemed complete by HPLC, water (130 L) was added to dissolve the byproduct salts. The mixture was filtered to remove the insoluble catalyst. The Pd—C cake was washed with fresh water (25 L). The filtrate was concentrated under reduced pressure, and toluene (105 L) was added. The mixture was made basic (pH=10) by the addition of aqueous potassium carbonate (70 L; K₂CO₃; 28.9 kg dissolved in 115 L of water). Methylene chloride (20 L) was then charged. The organic layer was separated, and sodium chloride (26.3 kg) was charged to the aqueous layer, which was back extracted with toluene (125 L). The combined organic phases were washed with potassium carbonate (45 L from above described aqueous potassium carbonate solution) and water (135 L), and the phases were separated. The organic phase was combined with toluene (110 L) and concentrated under vacuum, followed by another charge of toluene (110 L), which was again concentrated under vacuum. The drying was confirmed by an in-process testing (Karl Fisher). The resulting solution containing the title compound was used in the next step without further processing.

¹ HNMR (400 MHz, DMSO-d6): δ. 7.11 (s, 1H), 7.01 (br s, 2H), 6.31 (s, 1H), 3.97 (t, 2H), 3.69 (s, 3H), 3.57 (t, 4H), 2.42 (s, 3H), 2.44-2.30 (m, 6H), 1.91 (quin, 2H LC/MS Calcd for [M+H]⁺ 309.2, found 309.1.

### Preparation of 6-methoxy-7-(3-morpholin-4-yl-propoxy)-quinolin-4-ol dihydrochloride dehydrate

A solution of sodium ethoxide (98 L; 21% in ethanol) and ethyl formate (37 L) was added to the solution from the previous step. The solution was warmed to approximately 46° C. for approximately 3 hours. After the reaction was deemed complete by HPLC, water (100 L) was charged to the mixture, and the solution was made acidic (pH=1) by the addition of concentrated HCl (37%; 50 L) To the aqueous phase, acetone (335 L) was charged, and the mixture was cooled to approximately 10° C. and stirred for 5 hours, resulting in a slurry. The product was collected by filtration, and the product was washed with acetone (60 L) and dried under reduced pressure at approximately 40° C. The dried title compound (33.8 kg) was shown by HPLC to be 98% pure (percent area under the curve [AUC] by HPLC). Six lots of the title compound following procedure described were manufactured.

¹HNMR (400 MHz, DMSO-d6): δ. 11.22 (br s, 1H), 8.61 (d, 1H), 7.55 (s, 1H), 7.54 (s, 1H), 7.17 (d, 1H), 4.29 (t, 2 H), 3.99 (m, 2H), 3.96 (s, 3H), 3.84 (t, 2H), 3.50 (d, 2H), 3.30 (m, 2H), 3.11 (m, 2H), 2.35 (m, 2H), LC/MS Calcd for [M+H]⁺ 319.2, found 319.1.

### Preparation of 4-chlor-6-methoxy-7-(3 morpholin-4-yl)-quinoline

Phosphorous oxychloride (59.5 kg) was added to a solution of compound from the previous step (40.0 kg) in acetonitrile (235 L) that was heated to 50-55° C. When the addition was complete, the mixture was heated to reflux (approximately 82° C.) and held at that temperature with stirring for approximately 10 hours, at which time it was sampled for in-process HPLC analysis. The reaction was deemed complete when not more than 5% starting material remained. The reaction mixture was then cooled to 20-25° C., and methylene chloride was (100 L) charged. The resulting mixture was then quenched in pre-mixed methylene chloride (155 L), ammonium hydroxide (230 L), and ice (175 kg), while the temperature was maintained below 30° C. The resulting two-phase mixture was separated, and the aqueous layer was back extracted with methylene chloride (110 L). The combined methylene chloride phase was washed with water (185 L) and concentrated under vacuum (to a residual volume 40 L). This was used in the next step without further processing.

¹HNMR (400 MHz, DMSO-d6): δ. 8.61 (d, 1H), 7.56 (d, 1H), 7.45 (s, 1H), 7.38 (s, 1H), 4.21 (t, 2 H), 3.97 (s, 3H), 3.58 (m, 2H), 2.50-2.30 (m, 6H), 1.97 (quin, 2H) LC/MS Calcd for [M+H]⁺ 458.2, found 458.0.

### Preparation of 4-(2-fluoro-4-nitro-phenoxy)-6-methoxy-7-(3-morpholin-4-yl propoxy)quinoline

A solution of the product (from the previous step) and 2-fluoro-4-nitrophenol (16.8 kg) in 2,6-lutidine (55 L) was heated to approximately 160° C., with stirring, for approximately 3 hours, at which time it was sampled for in-process HPLC analysis. The reaction was considered complete with the conversion of compound from the previous step (>83%, HPLC). The reaction mixture was then cooled to approximately 75° C., and water (315 L) was added. Potassium carbonate (47.5 kg) dissolved in water (90 L) was added to the mixture, which was then stirred at ambient temperature overnight. The solids that precipitated were collected by filtration, and then washed with water (82 L). The wet solid was dissolved in methylene chloride (180 L) and aqueous potassium carbonate (65 L, 5%, by weight) was charged. After stirring for 0.4 hours, the phases were separated. This operation was repeated four times, and the resulting solution was concentrated under vacuum at 35° C. (residual volume, 40 L). T-butylmethylether (85 L) was then charged, and distillation continued under vacuum at 35° C. (residual volume, 50 L). This operation was repeated three times. The wet solid was then heated to approximately 52° C. in MTBE (70 L) for 0.3 hours. The solid was filtered and washed with MTBE (28 L). This operation was repeated twice. The wet solid was dried under vacuum at 35-45° C. under reduced pressure to afford 4-(2-fluoro-4-nitro-phenoxy)-6-methoxy-7-(3-morpholin-4-yl-propoxy) quinoline, the title compound (20.2 kg, 99% AUC). Two batches of the title compound were produced.

¹HNMR (400 MHz, DMSO-d6): δ. 8.54 (d, 1H), 8.44 (dd, 1H), 8.18 (m, 1H), 7.60 (m, 1H), 7.43 (s, 1H), 7.42 (s, 1H), 6.75 (d, 1H), 4.19 (t, 2H), 3.90 (s, 3H), 3.56 (t, 4H), 2.44 (t, 2H), 2.36 (m, 4H), 1.96 (m, 2H). LC/MS Calcd for [M+H]⁺ 337.1, 339.1, found 337.0, 339.0.

### Preparation of 3-fluoro-4-[6-methoxy-7-(3-morpholin-4-yl-propoxy)-quinolin-4-yloxy]-phenylamine

A reactor containing the product from the previous step (20.4 kg) and 10% palladium on carbon (50% water wet, 4.3 kg) in a mixture of ethanol (100 L) and water (87 L) containing concentrated hydrochloric acid (12.5 L) was pressurized with hydrogen gas (approximately 5 bar). The temperature of the reaction mixture was not allowed to exceed 46° C. When the reaction was complete, as evidenced by in-process HPLC analysis (typically 2 hours), the hydrogen gas was vented, and the reactor was inerted with nitrogen. The reaction mixture was filtered through a bed of Celite™ to remove the catalyst. Aqueous potassium carbonate (65 L, 5%) was charged to adjust pH (approximately pH 10). The resulting slurry was filtered and washed with water (63 L). The wet solid was suspended in acetonitrile (55 L) and water (55 L), and then the reaction mixture was stirred for approximately 0.3 hours. The solid was filtered and washed sequentially with water (35 L), acetonitrile (35 L), and toluene (35 L). The solid was suspended in toluene (100 L) and dried by azeotropic distillation. The azeotropic step was repeated three times. Finally, the toluene suspension was cooled, and the solids were filtered, washed with toluene (15 L), and dried at 40-45° C. under reduced pressure to afford the title compound (13.9 kg; 100% AUC). Two batches of the title compound were produced.

¹H NMR (400 MHz, DMSO-d6): δ. 8.45 (d, 1H), 7.51 (s, 1H), 7.38 (s, 1H), 7.08 (t, 1H), 6.55 (dd, 1H), 6.46 (dd, 1H), 6.39 (dd, 1H), 5.51 (br. s, 2H), 4.19 (t, 2H), 3.94 (s, 3H), 3.59 (t, 4H), 2.47 (t, 2H), 2.39 (m, 4H), 1.98 (m, 2H). LC/MS Calculated for [M+H]⁺ 428.2, found 428.1.

### Procedure for Direct Coupling

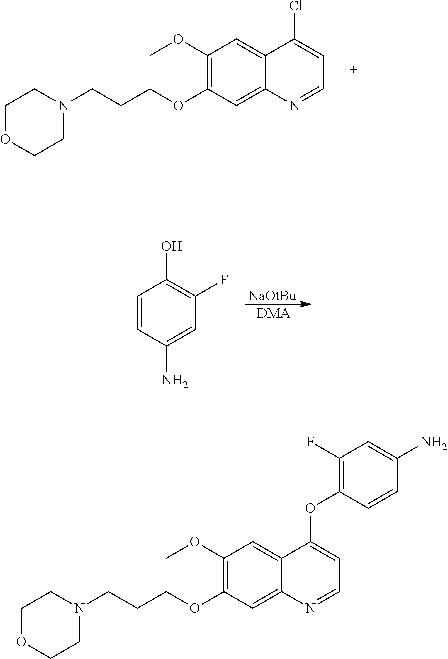

Solid sodium tert-butoxide (1.20 g; 12.5 mmol) was added to a suspension of the chloroquinoline (3.37 g; 10 mmol) in dimethylacetamide (35 mL), followed by solid 2-fluoro-4-hydroxyaniline. The dark green reaction mixture was heated at 95-100° C. for 18 hours. HPLC analysis showed about 18% starting material remaining and about 79% product. The reaction mixture was cooled to below 50° C., additional sodium tert-butoxide (300 mg; 3.125 mmol) and aniline (300 mg; 2.36 mmol) were added, and heating at 95-100° C. was resumed. HPLC analysis after 18 hours revealed less than 3% starting material remaining. The reaction was cooled to below 30° C., and ice water (50 mL) was added while maintaining the temperature below 30° C. After stirring for 1 hour at room temperature, the product was collected by filtration, washed with water (2×10 mL), and dried under vacuum on the filter funnel to yield 4.11 g of the coupled product as a tan solid (96% yield; 89%, corrected for water content). The ¹H NMR and MS were consistent with the desired product (97.8% LCAP; ˜7 wt % water by KF).

### Preparation of N-{3-Fluoro-4-[6-methoxy-7-(3-morpholin-4-yl-propoxy)-quinolin-4-yloxy]-phenyl)-N′-phenethyl-oxalamide

The compound from the previous step (13.7 kg), dimethyl formamide (70 L), and triethylamine (6.8 kg) were charged to a reactor. The reactor contents were cooled to approximately 5° C., and ethyl chlorooxoacetate (5.2 kg) was added so that the reaction temperature was maintained below 25° C. After the reaction was complete (typically 2-4 hours; determined by HPLC when <2% AUC compound from the previous step remained), a solution of 2-phenylethylamine (10.0 kg) in tetrahydrofuran (40 L) was charged to the reactor while maintaining the reaction temperature below 30° C. The reaction was deemed complete (typically complete in 2-4 hours) when <2% AUC ethyl ester remained by HPLC. The reactor contents were cooled to 20-25° C., and charged to a mixture of ice (44 kg), water (98 L), and ethanol (144 L) at a rate to maintain the temperature below 20° C. This was followed by stirring the reactor contents for at least 5 hours at 20-25° C., and the resulting slurry was concentrated under vacuum at 50° C. Water was then charged, and the resulting solid precipitate was recovered by filtration, washed with a mixture of ethanol (100 L) and water (100 L), and dried under vacuum at 60-65° C. to afford the title compound (16.9 kg; 98.7%, HPLC), which was used in the next step.

A second batch of this step was produced employing a similar methodology but resulted in lesser title compound. This was subjected to re-crystallization using the following strategy:

The title compound (17.2 kg) was suspended in THF (172 L) and water, heated to approximately 60° C. and was charged until complete dissolution was achieved. Ethanol (258 L) was then added and the mixture was cooled to approximately 25° C. and stirred for at least 8 hours. The resulting slurry was filtered, and the solid was washed with a mixture of ethanol/water (1:1, 168 L). The product was dried under vacuum at approximately 50° C. to yield the title compound (10.1 kg; 98.3%, HPLC).

¹H NMR (400 MHz, CDCl₃): δ. 9.37 (s, 1H), 8.46 (d, 1H), 7.81 (dd, 1H), 7.57 (t, 1H), 7.53 (s, 1H), 7.42 (s, 2H), 7.34-7.20 (m, 6H), 6.39 (d, 1H), 4.27 (t, 2H), 4.03 (s, 3H), 3.71 (m, 4H), 3.65 (q, 2H), 2.91 (t, 2H), 2.56 (br s, 4H), 2.13 (m, 2H); ¹³C NMR (100 MHz, d₆-DMSO): □160.1, 160.0, 159.5, 155.2, 152.7, 152.6, 150.2, 149.5, 147.1, 139.7, 137.3, 137.1, 129.3, 129.1, 126.9, 124.8, 117.9, 115.1, 109.2, 102.7, 99.6, 67.4, 66.9, 56.5, 55.5, 54.1, 41.3, 35.2, 26.4; IR (cm⁻¹): 1655, 1506, 1483, 1431, 1350, 1302, 1248, 1221, 1176, 1119, 864, 843, 804, 741, 700; LC/MS Calcd for (M+H): 603.66, found 603.

### Preparation of N-(3-Fluoro-4-[6-methoxy-7-(3-morpholin-4-yl-propoxy)-quinolin-4-yloxy]-phenyl)-N′-phenethyl-oxalamide bis phosphate

The compound from the previous step (16.8 kg) was charged to a reactor, and ethanol (170 L) was added. Phosphoric acid (10%, 72.6 kg) was added at a rate such that the batch temperature did not exceed 30° C. The batch was then heated to approximately 60° C. with stirring for 3 hours to ensure total dissolution. The batch was then cooled to 20-25° C. and stirred for approximately 6 hours, during which time the product precipitated. The solids were collected by filtration, washed twice with ethanol (152 L), and dried at 55-60° C. under vacuum to afford title compound (18.0 kg). A second batch of the title compound (9.9 kg) using similar strategy was produced.

¹H NMR (400 MHz, DMSO-d6): 11.04 (s, 1H), 9.14 (t, 1H), 8.48 (d, 1H), 8.04 (dd, 1H), 7.84 (br d, 1H), 7.55 (s, 1H), 7.50 (t, 1H), 7.46 (br s, 1H), 7.32 (m, 2H), 7.24 (m, 3H), 6.48 (d, 1H), 4.24 (br s, 2H), 3.96 (s, 3H), 3.74 (bs, 4H), 3.48 (q, 2H), 2.85 (m, 8H), 2.14 (br s, 2H).

All ranges disclosed herein are inclusive of the endpoints, and the endpoints are independently combinable with each other.

The use of the terms “a”, “an”, “the”, and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The foregoing disclosure has been described in some detail by way of illustration and example for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A pharmaceutical composition comprising:

| Ingredient | (% w/w) |
|---|---|
| Compound I | 31.68 |
| Microcrystalline Cellulose | 38.85 |
| Lactose anhydrous | 19.42 |
| Hydroxypropyl Cellulose | 3.00 |
| Croscarmellose Sodium | 3.00 |
| Total Intra-granular | 95.95 |
| Silicon dioxide, Colloidal | 0.30 |
| Croscarmellose Sodium | 3.00 |
| Magnesium Stearate | 0.75 |
| Total | 100.00 |

or

| Ingredient | (% w/w) |
|---|---|
| Compound I | 25.0-33.3 |
| Microcrystalline Cellulose | q.s |
| Hydroxypropyl Cellulose | 3 |
| Poloxamer | 0-3 |
| Croscarmellose Sodium | 6.0 |
| Colloidal Silicon Dioxide | 0.5 |
| Magnesium Stearate | 0.5-1.0 |
| Total | 100 |

or

| | Theoretical Quantity |
|---|---|
| Ingredient | (mg/unit dose) |
| Compound I | 100.0 |
| Microcrystalline Cellulose PH-102 | 155.4 |
| Lactose Anhydrous 60M | 77.7 |
| Hydroxypropyl Cellulose, EXF | 12.0 |
| Croscarmellose Sodium | 24.0 |
| Colloidal Silicon Dioxide | 1.2 |
| Magnesium Stearate (Non-Bovine) | 3.0 |
| Opadry Yellow | 16.0 |
| Total | 416 |

or

| Component | Weight/Weight Percent |
|---|---|
| Compound I | 25-29 |
| Microcrystalline Cellulose | q.s. |
| Lactose Anhydrous | 40-44 |
| Hydroxypropyl Cellulose | 2-4 |
| Croscarmellose Sodium | 2-8 |
| Colloidal Silicon Dioxide | 0.1-0.4 |
| Magnesium Stearate | 0.7-0.9 |
| Total | 100 |

wherein compound I is

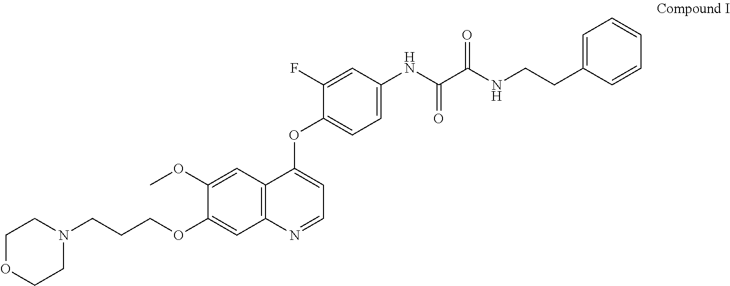 [SMILES: COc1cc2c(Oc3ccc(NC(=O)C(=O)NCCc4ccccc4)cc3F)ccnc2cc1OCCCN1CCOCC1]

2. (canceled)

3. (canceled)

4. A pharmaceutical composition comprising:
30-32 percent by weight of Compound I in at least one of the forms disclosed herein;
50-70 percent by weight of a filler;
2-4 percent by weight of a binder;
4-8 percent by weight a disintegrant; and
0.2-0.6 percent by weight of a glidant and 0.5-1 percent by weight of a lubricant.

5. The pharmaceutical composition of claim 4, wherein Compound I is the free base.

6. The pharmaceutical composition of claim 4, wherein Compound I is a pharmaceutically acceptable salt.

7. The pharmaceutical composition of claim 4, wherein Compound I is a hydrate.

8. The pharmaceutical composition of claim 4, wherein Compound I is in amorphous, substantially amorphous, crystalline, or substantially crystalline form.

9. (canceled)

10. The pharmaceutical composition of claim 4, wherein the filler is selected from the group consisting of sodium starch glycolate, corn starch, talc, sucrose, dextrose, glucose, lactose, xylitol, fructose, sorbitol, calcium phosphate, calcium sulfate, calcium carbonate, and microcrystalline cellulose, or mixtures thereof.

11. The pharmaceutical composition of claim 10, wherein the filler is a mixture of lactose and microcrystalline cellulose.

12. The pharmaceutical composition of claim 4, wherein the binder is selected from the group consisting of acacia, alginic acid, carbomer, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, and zein, or mixtures thereof.

13. (canceled)

14. The pharmaceutical composition of claim 4, wherein the disintegrant is selected from the group consisting of alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, powdered cellulose, pregelatinized starch, sodium alginate, and starch, or mixtures thereof.

15. (canceled)

16. The pharmaceutical composition of claim 4, wherein the gildant is colloidal silicon dioxide.

17. The pharmaceutical composition of claim 4, wherein the lubricant is selected from the group consisting of magnesium stearate, Lubritab®, stearic acid, and talc, or mixtures thereof.

18-27. (canceled)

28. The pharmaceutical composition of claim 1, further comprising a film coating.

29. The pharmaceutical composition of claim 1, wherein the film coating comprises Opadry Yellow.

30. The pharmaceutical formulation of claim 1 which is a tablet formulation.

31. A method for treating cancer, comprising administering to a patient in need of such treatment a pharmaceutical composition of claim 1 alone or in combination with another therapeutic agent.

32. The method of claim 31, wherein the cancer is selected from the group consisting of pancreatic cancer, kidney cancer, liver cancer, prostate cancer, gastric cancer, gastroesophageal cancer, melanoma, lung cancer, breast cancer, thyroid cancer, and astrocytic tumors.

33. The method of claim 32, wherein the cancer is pancreatic cancer, hepatocellular carcinoma (HCC), renal cell carcinoma, castration-resistant prostate cancer (CRPC), gastric or gastroesophageal junction cancer, melanoma, small cell lung cancer (SCLC), ovarian cancer, primary peritoneal or fallopian tube carcinoma, estrogen receptor positive breast cancer, estrogen receptor/progesterone receptor/HER2-negative (triple-negative) breast cancer, inflammatory (regardless of receptor status) breast cancer, non-small cell lung cancer (NSCLC), or medullary thyroid cancer.

34. A process for manufacturing a pharmaceutical composition comprising Compound I, comprising the steps of:
a. Delumping unmilled Compound I;
b. Premixing the delumped Compound I with Avicel PH102, lactose anhydrous 60M, and croscarmellose sodium to form a binder solution;
c. Wet high shear granulation of the binder solution to produce wet granules;
d. Wet screening of the wet granules to produce wet screened granules;
e. Fluid bed drying of the wet screened granules to produce dried granules;
f. Dry milling of the dried granules to produce dried milled granules;
g. Blending the dried milled granules with colloidal silicon and croscarmellose to produce an extragranular blend;
h. Lubricant blending of the extragranular blend and magnesium stearate to produce a final blend; and
i. Tablet compression of the final blend to form an uncoated core tablet.

35. The process of claim 34, further comprising the step of film coating of the uncoated core tablet.

36. A method for treating cancer, comprising administering to a patient in need of such treatment a pharmaceutical composition of claim 1, alone or in combination with another therapeutic agent.
